(19) United States
(12) Patent Application Publication
(10) Pub. No.: US 2015/0190234 A1
(43) Pub. Date: Jul. 9, 2015

(54) MIDLAY COMPARTMENTAL TIBIAL COMPONENT

(71) Applicants: Chih-Shing Wei, Lattingtown, NY (US); Peter S. Walker, New York, NY (US)

(72) Inventors: Chih-Shing Wei, Lattingtown, NY (US); Peter S. Walker, New York, NY (US)

(21) Appl. No.: 14/588,890

(22) Filed: Jan. 2, 2015

Related U.S. Application Data
(60) Provisional application No. 61/924,109, filed on Jan. 6, 2014.

(51) Int. Cl. A61F 2/38

(52) U.S. Cl. CPC A61F 2/389; A61F 2/3859

(57) ABSTRACT

A Midlay component includes a plastic bearing part and a metal tray part. The upper surface of the bearing part is dished in frontal and sagittal planes, to provide a bearing surface, such that in function, the femoral contact point will remain in the central region of the bearing surface. Hence the load-bearing on the plastic bearing part will be above the thickest portion in the central region. The metal tray part features a tray-bottom that enables retention of strong cortical and cancellous portion of a tibia in the treatment of osteoarthritis of a knee joint, and supports the plastic bearing part, with the rim of the metal tray part maintaining the position of the plastic bearing part and minimizing micromotions.

### FIELD OF THE INVENTION

The invention relates to a tibial resurfacing component, and more particularly to a Midlay compartmental tibial component, for resurfacing tibial bearing surfaces.

### BACKGROUND OF THE INVENTION

The overall field is the treatment of osteoarthritis of the knee joint, where the joint destruction is most commonly on the medial side of the knee. Since the early 1970's, unicompartmental knees (uni-knees) have been available for resurfacing the femoral and tibial bearing surfaces. The surgical procedure involves making facetted resections on the distal end of the femur, and a flat surface cut on the proximal tibia. The components are cemented in place. The results have been mixed, but with some component series showing only about 10% failure at 10 years. The most common failure modes of the components themselves have been loosening, wear and instability.

In recent years, burring techniques have been introduced whereby a pocket has been produced in the proximal tibia to fit an Inlay component, such that the component is cemented into the pocket thereby obtaining better cement pressurization and potentially better fixation. In addition, because the peripheral tibial soft tissues are preserved, there has been an absence of pain or discomfort which sometimes occurs with the standard Onlay components, for which a flat cut, without a pocket, is required. However, the Inlays were made of single-piece plastics, which deformed under load causing pain to the underlying bone, and also loosening due to bone resorption. Onlay components on the other hand have been primarily metal-backed, which distribute the load more uniformly. However because there is a minimum allowable thickness of plastic of approximately 6 mm, and at least 2 mm of metal-back thickness, the component thickness has required resection of more than the ideal depth of bone. This would apply if a plastic Inlay component was metal-backed also. Extra bone resection is especially a disadvantage because the density and strength diminishes rapidly with depth below the surface. Hence minimum resection is an advantage in preserving strong bone and enhancing durability of fixation.

### BRIEF SUMMARY OF THE INVENTION

The primary objective of the present invention is to provide an improved tibial component for use in the treatment of osteoarthritis of a knee joint.

Another objective of the present invention is to provide an improved tibial component that reduces the requisite resection of the proximal end of a tibia in the treatment of osteoarthritis of a knee joint.

Another objective of the present invention is to provide an improved tibial component that combines metal-backing advantages with retention of strong cortical portion of a tibia in the treatment of osteoarthritis of a knee joint.

Still another objective of the present invention is to provide an improved tibial component that incorporates sloped bearing surfaces such that the component can be seated at the prevailing varus angle of the medial tibia in the frontal plane.

A further objective of the present invention is to provide an improved tibial component that incorporates sloped bearing surfaces such that the component can be seated at the posterior slope in the sagittal plane.

Yet a further objective of the present invention is to provide an improved tibial component that incorporates a shape symmetry such that the component can be implanted on either the left or right knee.

### BRIEF DESCRIPTION OF DRAWINGS

FIG. 1 shows (A) an exploded, perspective view of an Inlay type of a tibial component and a tibia with a receiving pocket on the medial side, and (B) a perspective view of an implanted Inlay type of a tibial component;

FIG. 2 shows (A) an exploded, perspective view of an Onlay type of a tibial component and a tibia with a receiving platform on the medial side, and (B) a perspective view of an implanted Onlay type of a tibial component;

FIG. 3 is a perspective view, from the top, showing three types of tibial components;

FIG. 4 is a perspective view, from the bottom, showing three types of tibial components;

FIG. 5 is a perspective view, from the top, showing the resections on the medial side of the proximal tibia, to accommodate the three components shown in FIG. 4;

FIG. 6 is a perspective view, from the top, showing the volume of bone preservation attained by the present invention;

FIG. 7 shows a representative of a typical bone density distribution map taken from CT scans of a uni-patient;

FIG. 8 shows different views of a preferred embodiment of a Midlay component;

FIG. 9 shows a frontal section through the bone resections for an Onlay and a Midlay component;

FIG. 10 shows a sagittal section of the recess for a Midlay component;

FIG. 11 shows how the bearing surfaces of a Midlay component are constructed to allow for the slopes in the frontal and sagittal planes;

FIG. 12 shows the concept of maintaining the femoral contact points above the thickest portion in the central region of a Midlay component.

### DETAILED DESCRIPTION OF THE INVENTION

The present invention now will be described more fully hereinafter with reference to the accompanying drawings, in which some, but not all embodiments of the invention are shown. It is learned that after the description, any variation, modification or the like to the structure and the embodiments of the present invention is readily understood by any person skilled in the art. Thus, the following description is only for illustrative purpose only and does not, in any way, try to limit the scope of the present invention.

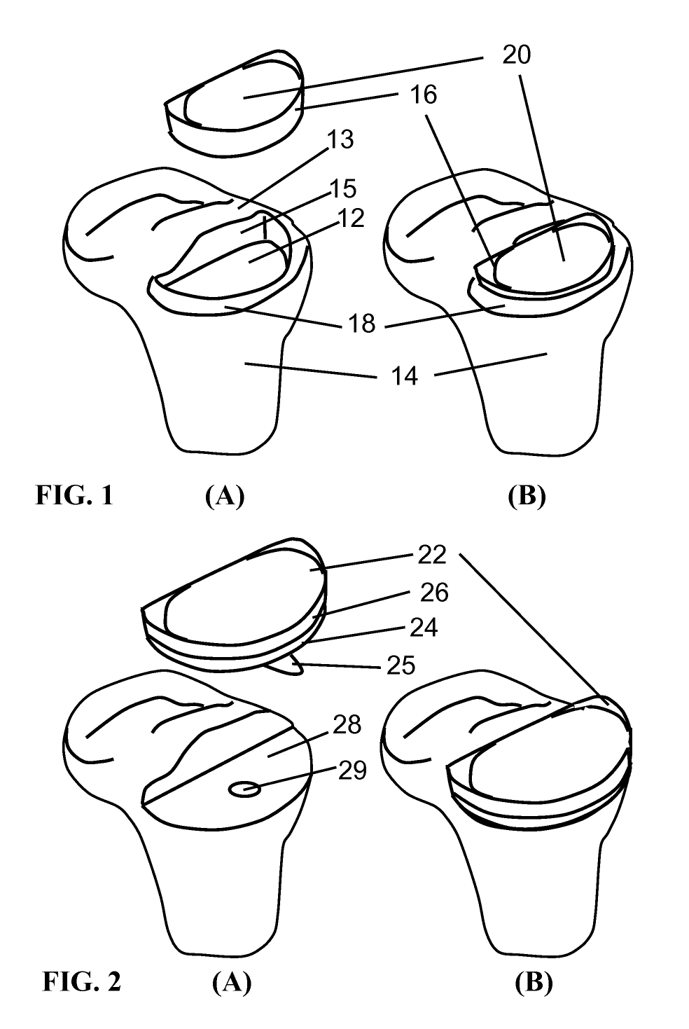

With reference to FIGS. 1A and 1B, it is noted that a recess pocket 12 is made on an upper surface 13 on the medial side of a tibia bone 14, and an Inlay component 16 is fitted inside the pocket 12, usually fixated with cement, but uncemented methods of fixation can be used. The Inlay component as shown has no metal-backing, consisting of a required load-bearing plastic of approximately 6 mm in thickness. A pocket rim 18 of approximately 3 mm of bone is preserved around the component 16, a value which can range from 2 to 4 mm. The seating on less than the complete upper surface 13 is compensated for by the shear fixation around the vertical walls 15 of the pocket 12, as well as the extra pressurization of the cement at surgery, obtaining better cement penetration into the cancellous structure. A dished upper surface 20 can be seen. This is more prominent, or taller, than the surrounding upper surface 13, because it is intended to replace the function of the meniscus (not shown) which effectively dishes the uncut upper surface 13.

FIGS. 2A and 2B show an Onlay component 22, consisting of a metal base 24 having a metal stem 25 at its bottom side and supporting a plastic bearing top 26. This is the most common type of component used today. The surgery is simple in that it only requires a single saw cut on the required plane 28 on the medial side and a burred recess 29 for receiving the stem 25. Excluding the stem 25, the typical thickness of an Onlay component is approximately 8 mm, including a required load-bearing plastic of approximately 6 mm and a metal backing of approximately 2 mm. A disadvantage of the design is that on cementing, the cement extrudes peripherally, reducing pressure in the cement layer.

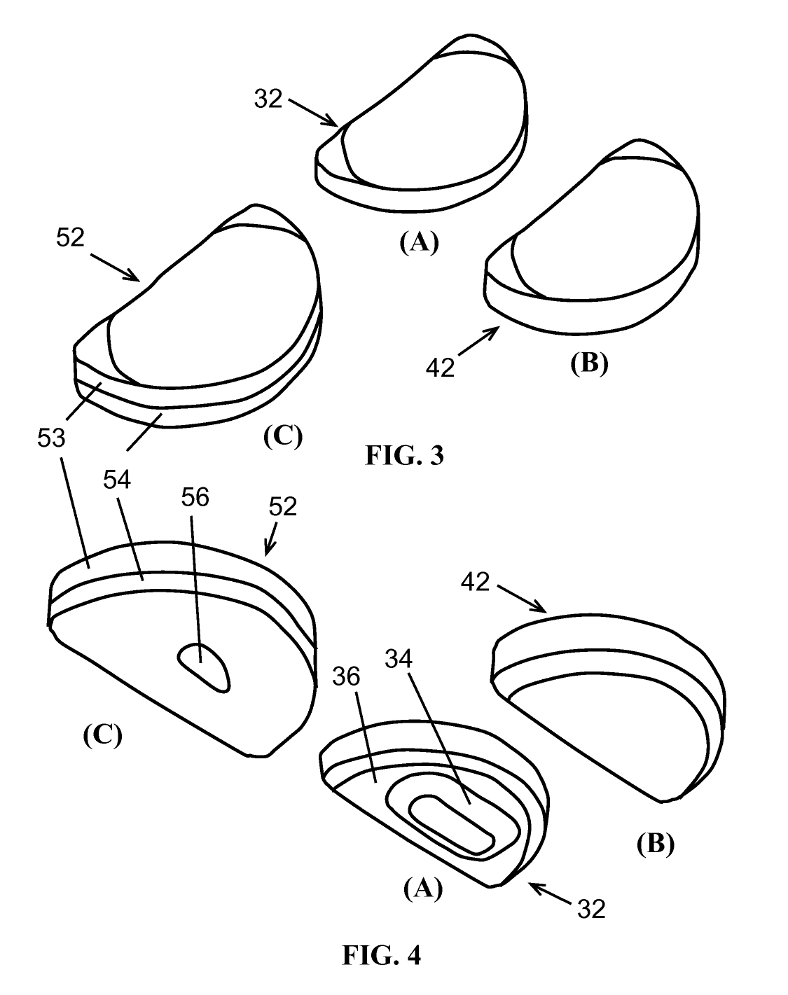

FIGS. 3 and 4 show three types of component which will be compared herein. All three types of component have a dished upper surface similar to the dished upper surface 20 shown in FIG. 1. The first is a preferred embodiment of the present invention, a Midlay component 32 (FIGS. 3A and 4A). A key feature is that, at its lower surface, only a central region 34 of the component 32 has a full required load-bearing thickness, the peripheral region 36 is approximately 2 mm thinner. Compared with the flat-bottom feature exhibited by the Inlay and Onlay components, this tray-bottom feature permits a surgeon to retain hard cortical bone and strong cancellous bone at the peripheral portion of a tibia during a knee replacement surgery. This embodiment may be made of a plastic or a metal. The second is an Inlay component 42 (FIGS. 3B and 4B), on which the full required load-bearing thickness extends over the entire component including its central and peripheral regions. The third is an Onlay component 52 having a plastic bearing top 53, a metal base 54 and a metal stem 56 (FIGS. 3C and 4C). The Onlay component 52 covers a larger surface area than the Midlay 32 and Inlay 42, but has a similar bearing surface and the flat bottom of the Inlay 42. Both the Inlay component 42 and the Onlay component 52 have a flat-bottom feature at their lower surfaces. The Midlay component 32 may be scaled to provide the functions of the Inlay component 42 and the Onlay component 52, while maintaining the advantage of retaining hard cortical bone and strong cancellous bone at the peripheral portion of a tibia during a knee replacement surgery.

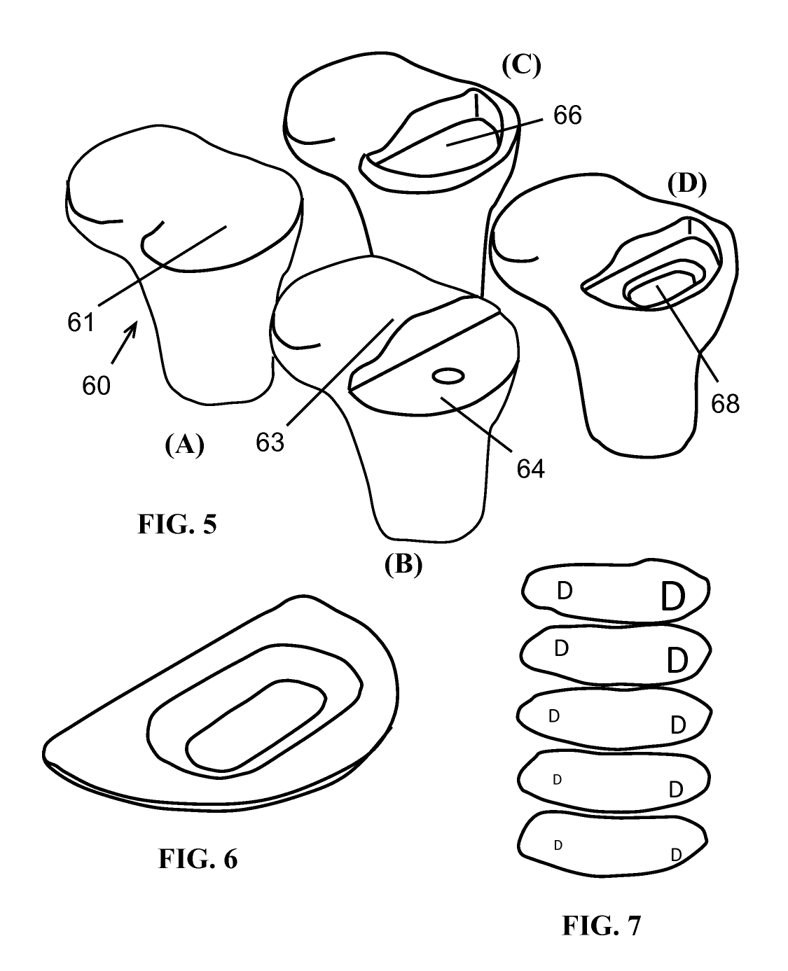

FIG. 5A shows an intact tibia 60. FIGS. 5B-5D show three resections on the proximal, medial side of the tibia 60, to accommodate the three types of component described above: resection 64 for the Onlay 52, recess 66 for the Inlay 42, and recess 68 for the Midlay 32. For the intact tibia 60, the cartilage wear and the collapse of the bone, usually result in a varus tilt of a few degrees in an arthritic bearing surface 61. The goal of the Midlay structure is to restore the original healthy bearing surface, which is hence elevated and angulated relative to the arthritic surface 61. For seating the Onlay 52, a resection 64 is created by bone cuts made horizontal to the long axis of the tibia 60, and vertical on the inner side of the plateau 63. The resected bone contains hard cortical bone and high density cancellous bone near the surface. The Inlay recess 66 may be tilted a few degrees in varus. For the Midlay recess 68, only the central region is at the full depth, the peripheral region being at approximately 2 mm less depth. This preserves more of the hard cortical bone and strong cancellous bone near the surface. The recess 68 may be tilted in varus. All of the bone cuts may be tilted in the sagittal plane approximately 7 degrees, to match the prevailing tibial slope.

FIG. 6 illustrates the volume of bone preservation attained by the Midlay component 32 with respect to the Inlay component 42, by subtracting the volume of the recess 68 for the Midlay component 32 from the volume of the recess 66 for the Inlay component 42. The thickest portions of these two components have approximately the same thickness. It is noted that the volume of such bone preservation with respect to the Onlay component 52 (not shown) is evidently greater than the one shown in FIG. 6. The practice of the tray-bottom feature as described above for the Midlay component can retain strong bone at the peripheral portion of a tibia; this tray-bottom feature and its bone-saving advantage can be extended to the resurfacing of other bones, such as femur, ulna, radius, or humerus. The bone-saving advantage of the tray-bottom feature illustrated for the medial side of a tibia, as shown in FIG. 6, can be expected when a Midlay component is used on the lateral side of a tibia, or on both the medial and lateral sides of a tibia.

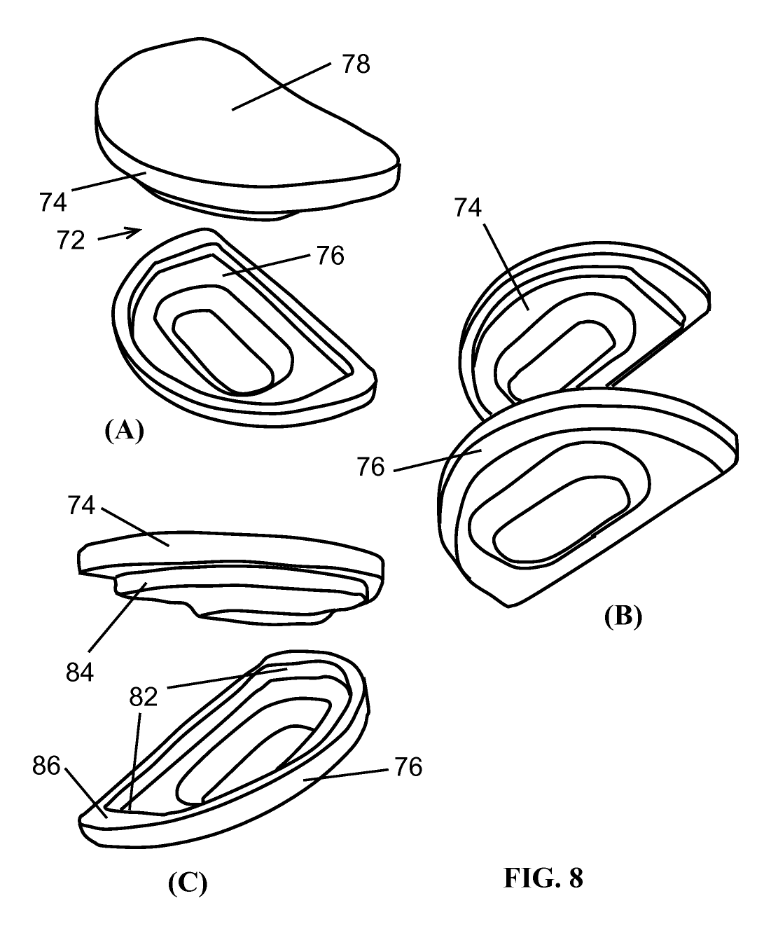

FIG. 7 shows a typical bone density distribution map taken from CT scans of a uni-patient, with the size of letter D representing the relative values in density. The bone density, and hence the strength, diminishes rapidly below the top surface of a tibia. Hence preservation of as much bone as possible is an advantage to provide maximum support to the component.

FIG. 8 shows different views of another preferred embodiment of a Midlay component 72. As shown in FIGS. 8A and 8B, the Midlay component 72 consists of two parts: a plastic bearing part 74 and a metal tray part 76. The upper surface of the bearing part 74 is dished in frontal and sagittal planes, to provide a bearing surface 78, such that in function, the femoral contact point will remain in the central region of the bearing surface 78. Hence the load-bearing on the bearing part 74 will be above the thickest portion in the central region, the required load-bearing thickness for plastic being approximately 6 mm. Positioned below the bearing part, the tray part provides a metal-backing of approximately 2 mm in thickness. The thickest portion in the central region is elongated in the anterior-posterior direction, because during a full flexion range, there is likely to be some displacements of the contact point occurring. It is noted that in the normal anatomic knee, the medial side is very stable and the contacts are similarly maintained centrally. Hence the dished component will restore the normal stability of the medial side. Once the correct size and thickness of a component have been determined at surgery, the plastic bearing part 74 may be snapped into the metal tray part 76. This is achieved with undercuts 82 in the metal tray part 76 and projections 84 in the plastic bearing part 74, as shown in FIG. 8C. A rim 86 of the metal tray part 76 also maintains the position of the plastic bearing part 74 in the metal tray part 76 and minimizes micromotions. The lower periphery of the metal tray part 76 is filleted to minimize stress concentrations. Alternatively, the Midlay component 72 may be insert-molded into a single-piece, metal-back Midlay component, or pre-assembled prior to surgery.

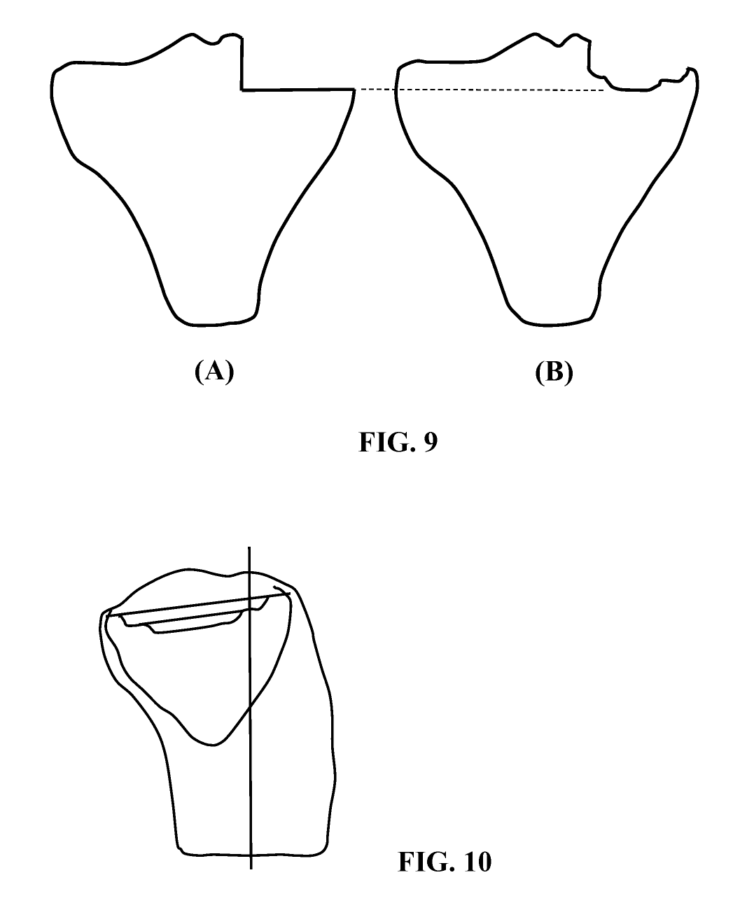

FIG. 9 shows a frontal section through the bone resections for the Onlay component 52 and the Midlay component 72. For the Onlay, the horizontal cut is shown in FIG. 9A at the full depth. For the Midlay, the reduced sectional cut can be seen in FIG. 9B. Also, the recess may be sloped at about 5 degrees medially, a varus inclination, to match the prevailing slope. This would further minimize the bone resection. The fillet radii at the edges of the recess can also be seen.

FIG. 10 shows a sagittal section of the recess for the Midlay component 72. This recess may be sloped at about 7 degrees, to match the prevailing slope of the particular tibia being treated.

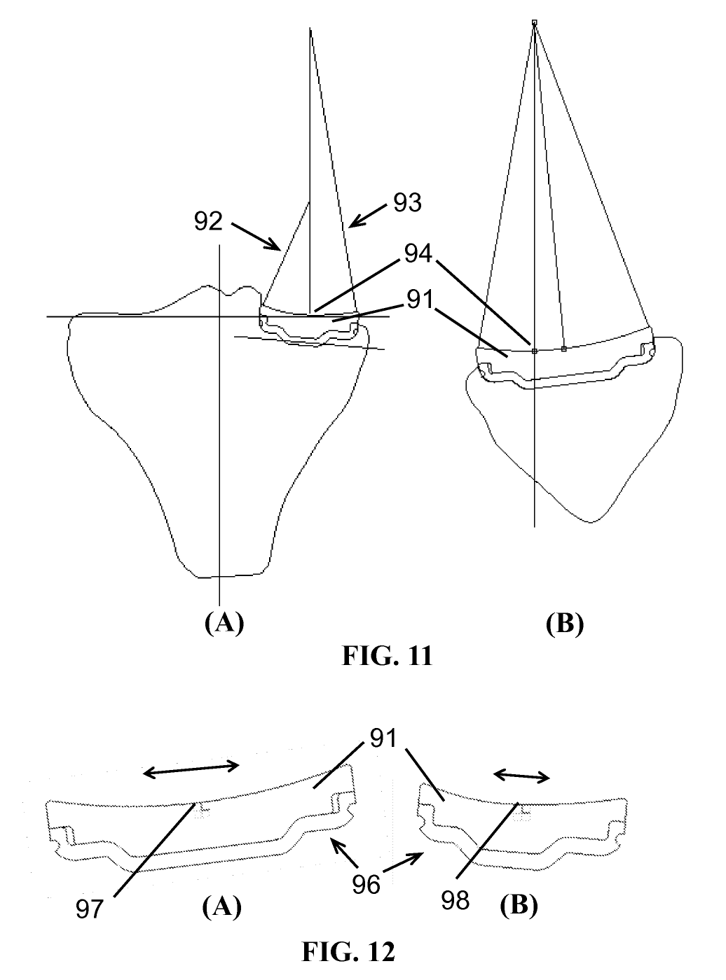

FIG. 11 shows how the bearing surface of a plastic bearing part 91 of a Midlay component may be constructed to allow for the slopes in the frontal and sagittal planes. In the frontal plane (FIG. 11A), even though there may be about 5 degrees medial tilt, the bearing surface is elevated such that the dwell point 94 (lowest point on the surface) is at approximately the normal half way across the medial plateau. The inner frontal radius 92 is also smaller than the outer frontal radius 93, to limit femoral motion towards the tibial spine where impingement could occur. In the sagittal plane (FIG. 11B), even though the bearing surface is symmetric, because of the posterior slope of the component, the dwell point 94 is more posterior than center, which is the normal situation. These features of radii mean that the same component can be used for right and left knees. In both views, the dwell point 94 is seen to be above the thickest portion in the central region of the plastic bearing part 91.

Related to FIG. 11, FIG. 12 shows specifically the concept of maintaining the femoral contact points above the thickest portion in the central region of the plastic bearing part 91 of a Midlay component 96. The radii shown in FIG. 11 are designed to be compatible with the typical slopes of the Midlay component 96 in the sagittal (FIG. 12A) and frontal (FIG. 12B) planes. Dishing of bearing surface, 97 and 98, in sagittal and frontal planes, respectively, maintain femoral contact points in central regions (indicated by the arrows) above the thickest portion in the central region of the plastic bearing part 91.

While the invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. In a bone resurfacing Midlay component wherein the component comprises:
a load-bearing dished upper surface;
a lower surface whereon a load-bearing central region of the component having a full thickness as required for load-bearing; and
a lower surface whereon a peripheral region of the component having a thickness less than that of the central region, enhancing bone retention.

2. The Midlay component as claimed in claim 1, wherein the upper surface of the component is dished in frontal and sagittal planes.

3. The Midlay component as claimed in claim 1, wherein the component is made of a plastic.

4. The Midlay component as claimed in claim 1, wherein the component is made of a metal.

5. The Midlay component as claimed in claim 1, wherein the component is used in resurfacing a tibia.

6. The Midlay component as claimed in claim 5, wherein the same component can be used for right and left knees.

7. The Midlay component as claimed in claim 1, wherein the component is used in resurfacing a femur.

8. The Midlay component as claimed in claim 1, wherein the component is scaled to provide the function of an Inlay component.

9. The Midlay component as claimed in claim 1, wherein the component is scaled to provide the function of an Onlay component.

10. The Midlay component as claimed in claim 1, wherein the load-bearing dished upper surface is provided by a bearing part and the lower surface is provided by a tray part.

11. The Midlay component as claimed in claim 10, wherein the bearing part is made of a plastic and the tray part is made of a metal.

12. The Midlay component as claimed in claim 11, wherein the bearing part provides a bearing surface such that the femoral contact point remains in a central region of the bearing surface and the load-bearing on the bearing part is above the thickest plastic in the central region.

13. The Midlay component as claimed in claim 10, wherein the bearing part and the tray part are scaled to provide the function of an Inlay component.

14. The Midlay component as claimed in claim 10, wherein the bearing part and the tray part are scaled to provide the function of an Onlay component.
